(19) United States
(12) Patent Application Publication
(10) Pub. No.: US 2015/0025350 A1
(43) Pub. Date: Jan. 22, 2015

(54) Electrode With Guide Tunnel for a Cannula, and Kit Comprising Electrode and Cannula

(71) Applicant: BIOTRONIK SE & Co. KG, Berlin (DE)

(72) Inventor: Christian Schnittker, Berlin (DE)

(21) Appl. No.: 14/303,779

(22) Filed: Jun. 13, 2014

Related U.S. Application Data
(60) Provisional application No. 61/856,061, filed on Jul. 19, 2013.

(51) Int. Cl. A61B 5/00; A61N 1/05

(52) U.S. Cl. CPC A61B 5/6882; A61N 1/05; A61B 5/6869
USPC 600/373; 607/116; 606/129

(57) ABSTRACT

An electrode for conducting electrical signals from or to an organ, and also to a kit for examining or treating an organ, said kit including an electrode that can be anchored to the organ. In order to reduce the risk of inflammation of the organ caused by the treatment or examination thereof, the electrode is formed in accordance with the present disclosure with a guide tunnel for a cannula for the administration of an active ingredient.

### CROSS-REFERENCE TO RELATED APPLICATIONS

This patent application claims the benefit of co-pending U.S. Provisional Patent Application No. 61/856,061, filed on Jul. 19, 2013, the disclosure of which is hereby incorporated by reference in its entirety.

### TECHNICAL FIELD

The present invention relates to an electrode for conducting electrical signals from or to an organ or other bodily tissue, comprising a connector end for connection of the electrode to a device receiving or emitting the signals, and also comprising an anchoring end, by means of which the electrode can be anchored to the organ in a signal-conducting manner. The present invention also relates to a kit for examining or treating an organ, said kit comprising an electrode that can be anchored to the organ.

### BACKGROUND

In order to bring the electrode into contact with the organ or other bodily tissue, the electrode is generally introduced into the body of a patient. A stable connection between the electrode and the organ is often achieved by inserting the anchoring end at least partially into the organ. The body and even the organ are thus injured in order to examine and/or treat the organ. The injury includes, for example, a risk of inflammation, wherein, in the case of known electrodes, effects of the inflammation are reduced by coating the anchoring end with an active ingredient inhibiting the inflammation. Alternatively, an active ingredient reservoir, for example, a silicone ring releasing the active ingredient over time, can be attached onto the anchoring end and delivers the active ingredient to the organ.

The quantities of active ingredient deliverable with the known electrodes are so low, however, that the inflammation cannot be suppressed sufficiently.

An object of the present invention is therefore to provide an electrode and a kit of the type mentioned in the introduction (Technical Field), with which the risk posed for a patient during the examination and/or treatment of the organ is considerably reduced.

The present invention is directed toward overcoming one or more of the above-identified problems.

### SUMMARY

For the electrode mentioned in the introduction, at least the above object is achieved by a guide tunnel for a cannula, said guide tunnel extending at least in part through the electrode. For the kit mentioned in the introduction, at least the above object is achieved in that the electrode is an electrode according to the present invention and the kit comprises a cannula, wherein the cannula is embodied so as to be flexible and insertable into the guide tunnel.

Due to the provision of the guide tunnel in the electrode, the cannula can be slid into the electrode and the active ingredient can be conducted through the electrode as far as the organ without the need to perform a further intervention. A required quantity of active ingredient can be supplied to the organ, wherein the quantity of active ingredient is not restricted by the known administration mechanisms.

In particular, a large quantity of the active ingredient can be brought in a target-oriented manner to the site of the electrode fixing. Here, the active ingredient can be designed to remain at the site of the injection over a number of days or even weeks, and to be released little by little during this residence period. The long residence period of the active ingredient at the injection site can be achieved, for example, by using special galenic formulations or active ingredient derivatives. Here, a release period of the active ingredient located at the injection site from one to four weeks is often desired. The active ingredient thus remains at the injection site for a long period and is released there over time. The residence period, or release period, can be achieved by the provision of the active ingredient in, for example, microparticles or nanoparticles, microemulsions or liposomes and also by adaptations of the molecular structure of the active ingredient (for example, fatty acid esters that are not easily soluble).

Alternatively, or in addition to active ingredients that are intended to suppress inflammation, other active ingredients, such as, for example, non-steroidal antirheumatics, antiarrythmics or similar active ingredients and also stem cells, can be injected directly into the organ to be examined and/or treated and, for example, intracardially in a simple and efficient manner. These active ingredients can also be injected selectively by means of the electrode according to the present invention and in relative large quantities, and can be released over a long period of up to a number of weeks.

A solution according to the present invention can be further improved by different embodiments, which can each be combined advantageously with one another arbitrarily. These embodiments and the advantages associated therewith will be discussed hereinafter.

In a first advantageous embodiment, the guide tunnel may extend as far as the anchoring end. The guide tunnel preferably opens in a longitudinal direction of the electrode pointing from the connector end to the anchoring end. The guide tunnel can thus guide the active ingredient reliably as far as the anchoring end and, therefore, as far as the organ.

The electrode can be embodied at least in some portions as a guide catheter for the cannula, whereby the cannula can be easily handled.

In order to apply the active ingredient, the cannula, in its application position, can protrude beyond the anchoring end in the longitudinal direction of the electrode. In particular, the active ingredient can be delivered hereby into areas of the organ that are arranged further within the organ than the depth reached by the anchoring end in the organ. The deeper delivery of the drug allows an administration of a larger quantity of the drug compared to a delivery over the surface. Alternatively, or additionally, the active ingredient can be injected directly into the area of the organ injured by the electrode in order to inhibit the inflammation even more effectively. The cannula can thus extend in its application position in the longitudinal direction at least as far as or even beyond the anchoring end of the anchoring coil.

The guide tunnel can extend from the anchoring end as far as the connector end and, therefore, the guide tunnel can be easily accessible for an operator of the electrode and the cannula can be easily insertable into the guide tunnel. The cannula can then even be inserted into the electrode when the anchoring end is anchored to the organ. The cannula is preferably longer than the electrode.

The anchoring end can be formed with an anchoring coil, which runs around the guide tunnel. The anchoring end therefore also guides the cannula without the cannula being able to collide with the anchoring end. The cannula, in the application position, preferably protrudes beyond the anchoring coil in the longitudinal direction. In the application position, the cannula can thus protrude out from the anchoring end of the electrode in the longitudinal direction.

If the anchoring end and, in particular, the anchoring coil thereof penetrates the organ, the organ thus does not need to be injured again in other areas by the cannula in order to administer the drug, since the cannula can be introduced into the organ in a anchoring position, in which the anchoring end anchors the electrode to the organ.

A free end of the anchoring coil can be formed as a piercing tip, which is oriented substantially perpendicularly to the longitudinal direction of the electrode. An anchoring end of this type allows the electrode to be anchored to the organ by means of, for example, a simple screwing motion, wherein greater injuries to the organ are avoided.

The anchoring coil is preferably connected electrically and conductively to the connector end, such that the anchoring coil can contact the organ in a signal-transmitting manner with low loss, and it is possible to dispense with additional contacting elements.

The electrode may be an electrode, which can be anchored to a cardiac wall, for a cardiac pacemaker or defibrillator, for example. In particular, the electrode can be designed to be arranged permanently in the body of a patient whose organ is to be examined and/or treated. For example, the electrode can be implanted permanently with the cardiac pacemaker or defibrillator in the patient. The administration of the active ingredient by means of the cannula inserted into the electrode can be performed during or after the anchoring of the electrode to the organ. After the administration of the drug, the cannula is preferably removed and the electrode is connected to the device and, for example, to the cardiac pacemaker or the defibrillator, and is then implanted fully together with the device. The device, together with the electrode and the cannula, can be a component of the kit. The signals that can be conducted by the electrode can be measurement signals representing a condition of the organ and/or measurement signals generated by the organ and/or may comprise treatment signals generated by the device for treatment of the organ.

The administered active ingredient may form an active ingredient reservoir in the organ, said reservoir releasing the active ingredient over time and, for example, over a number of days or weeks.

Further features, aspects, objects, advantages, and possible applications of the present invention will become apparent from a study of the exemplary embodiments and examples described below, in combination with the Figure, and the appended claims.

### DESCRIPTION OF THE DRAWING

The present invention will be explained by way of example hereinafter on the basis of an exemplary embodiment with reference to the drawing, in which:

FIG. 1 shows a schematic illustration of an exemplary embodiment of the electrode according to the present invention with a cannula inserted into the electrode.

### DETAILED DESCRIPTION

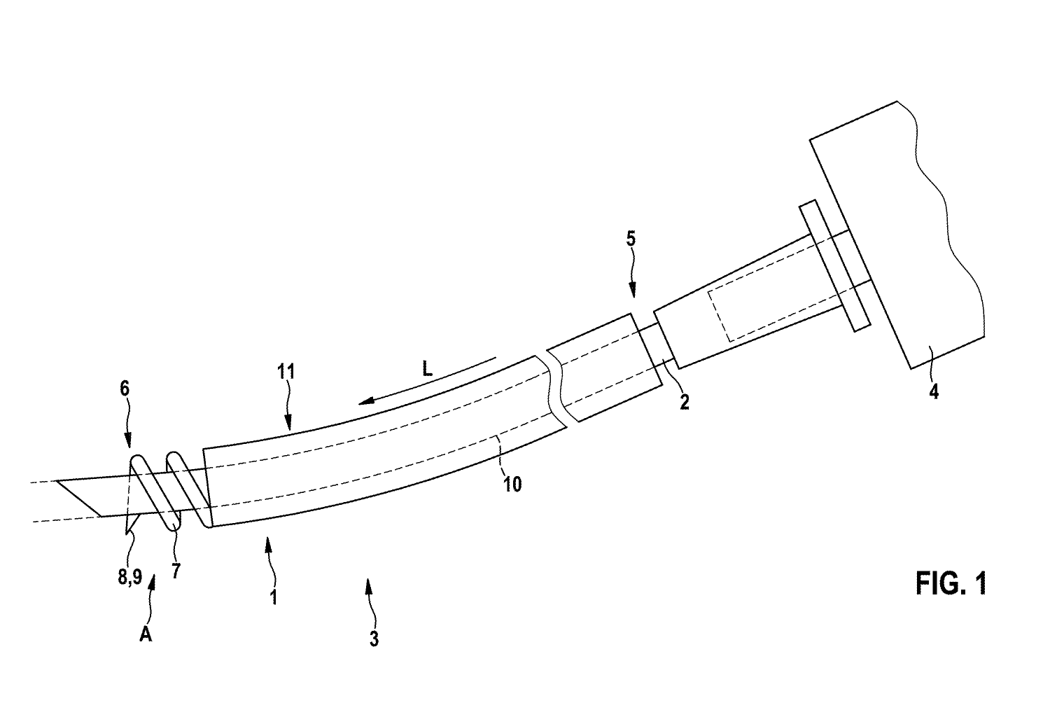

FIG. 1 shows the electrode 1 according to the present invention with a cannula 2 guided through the electrode 1 for administration of an active ingredient. The electrode 1 and the cannula 2, either in the illustrated assembled state or separately from one another, can be part of a kit 3 for the examination or treatment of an organ. The cannula 2 is illustrated ready for use in the exemplary embodiment, and is fluidically connected to a syringe 4, which provides the active ingredient. The active ingredient contained in the syringe 4 can be released to the organ through the electrode 1 and the cannula 2. The syringe 4 and/or the active ingredient may be components of the kit 3.

The electrode 1 is formed with a connector end 5 for connection of the electrode 1 to a device emitting or receiving signals. In the exemplary embodiment in FIG. 1, the connector end 5 is formed in a longitudinal direction “L” of the electrode as a first portion of the electrode 1. An anchoring end 6 of the electrode 1, arranged opposite the connector end 5 along the electrode 1, is designed for the anchoring of the electrode 1 to the organ, for example, by means of an anchoring coil 7, which can be screwed into the organ. The anchoring coil 7 is substantially helical and is illustrated with a free end 8. The free end 8 is formed as a piercing tip 9, which is oriented so as to be pointing substantially perpendicularly to the longitudinal direction L. Since the anchoring coil 7 extends along the helix shape, the organ is not excessively injured as the anchoring end 6 is screwed in.

The anchoring end 6 and, in particular, the anchoring coil 7 is preferably connected to the connector end 5 in a signal-conducting and, for example, in an electrically conductive manner, such that signals can be conducted back and/or forth between a device connected to the connector end 5 and the anchoring end 6 during the examination and/or treatment process.

A guide tunnel 10 for the cannula 2 can extend through the electrode 1 between the connector end 5 and the anchoring end 6. In the exemplary embodiment in FIG. 1, the cannula 2 is inserted into the guide tunnel 10 and extends from the connector end 6 in the longitudinal direction L beyond the piercing tip 9 of the anchoring end 6. The anchoring coil 7 runs around the guide tunnel 10, such that the cannula 2 can be inserted into the electrode 1 not only within a tubular portion 11 of the electrode 1, but also in the region of the anchoring coil 7 in a manner guided by the guide tunnel 10. The guide tunnel 10 extends here along the longitudinal direction L, preferably completely from the connector end 5 to the anchoring end 6 through the electrode 1.

Along the longitudinal direction L, the cannula 2 preferably has a length that is greater than a length of the electrode 1 between the connector end 5 and the anchoring end 6 or the piercing tip 9. In the exemplary embodiment in FIG. 1, the cannula 2 is illustrated in its application position A, in which an active ingredient can be delivered to an organ through the cannula 2 when the anchoring end 6 is anchored to the organ and, for example, the anchoring coil 7 protrudes at least in part into the organ. In the application position A, the cannula 2 extends in the longitudinal direction L beyond the anchoring end 6. In particular, the cannula 2, in its application position A, can protrude beyond the anchoring coil 7 in the longitudinal direction L.

In order to bring the electrode 1 through the body of a patient and into contact with the organ, the electrode 1 has a flexible structure. So that the cannula 2 can also be guided as far as the organ, the cannula 2 may have a flexible structure comparable to that of the electrode 1. Here, the cannula 2 can be designed to be inserted in a curved state of the electrode 1 into the guide tunnel 10. Alternatively, the cannula 2 arranged in the guide tunnel 10 can be bendable together with the electrode 1.

The electrode 1 is, for example, an electrode 1, which can be anchored to a cardiac wall, for a cardiac pacemaker or defibrillator, for example. In particular, the electrode 1 is designed to be arranged permanently in the body of a patient whose organ is to be examined and/or treated. For example, the electrode 1 can be implanted in the patient permanently with the cardiac pacemaker or defibrillator. The administration of the active ingredient through the cannula 2 inserted into the electrode 1 can be performed during or after the anchoring of the electrode 1 to the organ. After the administration of the drug, the cannula 2 is preferably removed and the electrode 1 is connected to the device and, for example, to the cardiac pacemaker or the defibrillator, and is then implanted fully together with the device.

The administered active ingredient can form an active ingredient reservoir in the organ, said reservoir releasing the active ingredient over time and, for example, over a number of days or weeks.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/we claim:

1. An electrode for conducting electrical signals from or to an organ, said electrode comprising:
a connector end for connection of the electrode to a device receiving or emitting the electrical signals; and
an anchoring end, by means of which the electrode can be anchored in a signal-conducting manner to the organ, wherein the electrode includes a guide channel for a cannula, said guide channel extending at least in part through the electrode.

2. The electrode as claimed in claim 1, wherein the guide channel extends as far as the anchoring end.

3. The electrode as claimed in claim 1, wherein the guide tunnel ex-tends from the anchoring end as far as the connector end.

4. The electrode as claimed in one of claim 1, wherein the anchoring end comprises an anchoring coil, which runs around the guide tunnel.

5. The electrode as claimed in claim 4, wherein a free end of the anchoring coil is formed as a piercing tip, which is oriented so as to be pointing substantially perpendicularly to a longitudinal direction of the electrode.

6. The electrode as claimed in claim 4, wherein the anchoring coil is electrically and conductively connected to the connector end.

7. A kit for examining or treating an organ, said kit comprising:
an electrode that can be anchored to the organ, wherein the electrode is an electrode as claimed in claim 1, and the kit further comprises a cannula, wherein the cannula is flexible and can be inserted into the guide tunnel.

8. The kit as claimed in claim 7, wherein the electrode is embodied as a guide catheter for the cannula.

9. The kit as claimed in claim 7, wherein the cannula, in its application position, protrudes beyond the anchoring coil in a longitudinal direction.

10. The kit as claimed in claim 7, wherein the cannula, in its application position, extends in a longitudinal direction beyond the anchoring end of the anchoring coil.
